(19) United States
(12) Patent Application Publication
(10) Pub. No.: US 2004/0053410 A1
(43) Pub. Date: Mar. 18, 2004

(54) Method for introducing a nucleic acid into a cell (transfection) by means of calcium phosphate and transfected cell

(76) Inventors: Markus Horer, Martinsried (DE); Bernd Rehberger, Gersthofen (DE); Ulrich Moebius, Gauting (DE)

Correspondence Address: CLARK & ELBING LLP, 101 FEDERAL STREET, BOSTON, MA, 02110, US

(21) Appl. No.: 10/276,207

(22) PCT Filed: May 15, 2001
(86) PCT No.: PCT/EP01/05531

(30) Foreign Application Priority Data
May 17, 2000 (DE) 100-24-334.07

(51) Int. Cl. C12N 15/85

(52) U.S. Cl. 435/455

(57) ABSTRACT

The invention relates to a method for introducing a nucleic acid into at least one cell by means of calcium phosphate (CaPi), to a method for producing a transfected cell and to the transfected cell as such.

The present invention relates to a method for introducing nucleic acid into at least one cell (transfection) using calcium phosphate (CaPi), with the nucleic acid initially being mixed with a reaction solution which contains Ca²⁺ and PO₄³⁻ ions, the reaction solution subsequently being incubated, resulting in the formation of a precipitate which at least contains the nucleic acid, calcium and phosphate, the precipitate subsequently being added to the cell and the cell being incubated once again, resulting in the nucleic acid being taken up into the cell. In addition, the present invention relates to a method for producing the transfected cell.

With interest in elucidating the structure and function of genes and proteins within the context of genetic manipulation and biotechnology increasing, a very wide variety of methods for introducing nucleic acids into freshly isolated cells or into cultured cells have been developed in laboratories throughout the world. Depending on the problem to be investigated, cells can be treated with externally added nucleic acid (external nucleic acid) and either integrate this nucleic acid permanently into the genome or have it as a circular, extrachromosomal nucleic acid molecule. In both cases, the external nucleic acid is replicated prior to cell division and passed on to the daughter cell, for which reason this transfection method is also termed stable transfection. Alternatively, cells may only take up an external nucleic acid transiently (transient transfection), with the nucleic acid which has been supplied not replicating and therefore not being selectively passed on to the daughter cells in connection with the subsequent cell divisions and, depending on the copy number, being lost more or less rapidly. As a rule, transiently transfected cells are disrupted (harvested or lyzed) a few hours or days after the transfection. Following on from this, the functions of the proteins which were encoded on the external nucleic acid and have been expressed in the cell following the transfection, are, for example, investigated in further laboratory experiments.

Irrespective of the method which is used for introducing nucleic acid into the cell, the efficiency depends very greatly, both in the case of stable transfection and in the case of transient transfection, on the cell type employed. Some cells which have already been in culture for a relatively long period of time (cell lines) differ many fold in their ability to take up external nucleic acid and to express the proteins which are encoded on this nucleic acid. Thus, a transfection method which has been established for one cell type, and which leads to good transfection efficiencies, may be completely unsuitable for another cell type.

One of the oldest methods for introducing nucleic acids into cells makes use of the polysaccharide DEAE dextran as the substance for carrying external nucleic acid. DEAE dextran was originally used as a mediator for introducing polio virus ribonucleic acid (RNA) (Vaheri and Pagano (1965) Virology 27:434), and simian virus (SV40) and polyomavirus deoxyribonucleic acid (DNA) (McCutchan and Pagano (1968) J. Natl. Cancer Inst. 41:351; Warden and Thorne (1968) J. Gen. Virol. 3:371) into cells. The method is still used, even today, with minor modifications, for transfecting viral genomes and circular DNA molecules (plasmids) which carry viral gene sequences. Although the precise mechanism of action of DEAE dextran is not known, it is assumed that the polymer binds the external nucleic acid, in most cases external DNA, and in this way prevents attack by cellular nucleic acid-destroying enzymes (nucleases) and/or itself binds to the surface of cells and thereby promotes the uptake of DNA into the cell by means of endocytosis. DEAE dextran-mediated transfections are normally used exclusively for transiently expressing cloned genes and not for stably transfecting cells. While some established cell lines, such as BSC-1, CV-1 and COS, can be transfected very well using DEAE dextran, other established cell lines, such as HeLa cells, are unsuitable, probably due to the toxicity of the polymer. As a rule, small quantities of external DNA are used for transfections employing DEAE dextran, with maximum efficiencies for transfecting 10⁵ cells having been obtained with only 100-200 ng of plasmid DNA. Larger quantities of DNA (<2-3 μg) exhibit an inhibitory effect rather than an increased effect. In contrast to other transfection methods, which are also described below and which require high concentrations of DNA, no additional carrier DNA, such as salmon sperm DNA or herring sperm DNA, is added to the external DNA, which is to be transfected, in the case of DEAE dextran-mediated transfection. Although this transfection method was developed more than 30 years ago, it is in most cases carried out using only minor modifications of the method which was originally described. However, since then, it has become known that both the concentration of the DEAE dextran employed, and the time for which the cells are exposed to the DNA/DEAE dextran mixture (incubation time), are critical for the success of the transfection.

Since not all established cell lines can be efficiently transfected using DEAE dextran, alternative transfection methods have been developed. Another method uses the polycation polybrene as mediator, in particular for introducing DNA into cells which have proven to be resistant toward DEAE dextran (Kawai and Nishizawa (1984) Mol. Cell. Biol. 4:1172; Chaney et al. (1986) Somatic Cell Mol. Genet. 12:237). In contrast to DEAE dextran-mediated transfection, polybrene is suitable for stably transfecting cells as well as for transiently transfecting them. However, this transfection method is restricted to nucleic acids which are of low molecular weight, for example plasmid DNA.

In addition, cloned DNA can be introduced into cultured cells by fusing the cells with protoplasts which are obtained from bacteria which harbor the plasmid DNA of interest (Schaffner (1980) Proc. Natl. Acad. Sci. 77:2163; Rassoulzadegan et al. (1982) Nature 295:257). For this method, bacteria are cultured in the presence of chloramphenicol in order to amplify the plasmid DNA which the bacteria harbor internally in high copy number. Subsequently, the bacteria are treated with the enzyme lysozyme, resulting in their cell wall being destroyed. The protoplasts which are obtained in this way are mixed with the cells to be transfected, with the polymer polyethylene glycol (PEG) being added in order to promote the fusion of cell and bacterial protoplasts. During this process, both the plasmid DNA and the DNA of the bacterial chromosome gain access to the cell to be transfected, with the plasmid DNA being conveyed into the nucleus of the cell in a manner which has not yet been elucidated. Subsequently, the PEG is removed and the cells are treated with fresh cell culture medium which contains an antibiotic, as a rule kanamycin, for the purpose of preventing surviving bacteria from growing. The method of protoplast fusion has been used both for transiently expressing cloned genes and for establishing mammalian cell lines. When bacteria and mammalian cells were mixed in a ratio of 10 000:1, mammalian cells were transfected with an efficiency of approx. 6% (Schaffner (1980) see above). This indicates that, while the method of protoplast fusion is relatively inefficient in comparison with other transfection methods, it is particularly suitable for introducing DNA into cell lines which are resistant to the DEAE dextran-mediated endocytosis of DNA. It has furthermore been observed that protoplast fusion very frequently leads to the integration of several copies of the plasmid DNA into the host chromosome (Robert de Saint Vincent et al. (1981) Cell 27:267).

The use of a short electrical current impulse of high voltage has also been employed for introducing DNA into a large number of bacteria and plant and animal cells (Neumann et al. (1982) EMBO J. 1:841; Wong and Neumann (1982) Biochem. Biophys. Res. Commun. 107:584; Potter et al. (1984) Proc. Natl. Acad. Sci. 81:7161; Fromm et al. (1985) Proc. Natl. Acad. Sci. 82:5824, Fromm et al. (1986) Nature 319:791; Andreason and Evans (1988) BioTechniques 6:650). This transfection method is termed electroporation (Neumann et al. (1982) see above). While this method is suitable both for transiently transfecting and stably transfecting cells, some of the transfection efficiencies which are achieved in the individual reports differ many times over. The current impulse produces extremely small pores, in the order of size of a few nanometers, in the plasma membrane of the cell to be transfected (Neumann et al. (1982) see above; Zimmermann (1982) Biochim. Biophys. Acta 694:227). The external DNA is taken up into the cytoplasma of the cell either directly through these pores or as a consequence of a reorganization of the membrane components which accompanies the reclosing of the pores which have arisen. In contrast to DEAE dextran-mediated transfection or protoplast fusion, electroporation as a rule leads to cell lines which harbor one copy of the external DNA and only occasionally to cell lines which harbor several copies (Boggs et al. (1986) Exp. Hematol. 14:988). In the particular case of electroporation, the transfection efficiency is influenced by a large number of factors. In this connection, the strength of the electrical field which is applied (Patterson (1979) Methods Enzymol. 58:141), the length of the electrical impulse (Rabussay et al. (1987) Bethesda Res. Lab. Focus 9(3):1), the temperature (Reiss et al. (1986) Biochem. Biophys. Res. Commun. 137:244; Chu et al. (1987) Nucleic Acids Res., 15:1311), the conformation and concentration of external DNA (Neumann et al. (1982) see above; Potter et al. (1984) see above), and the ionic composition of the medium (Rabussay et al. (1987) see above), have been recognized as being particularly critical. Although it may sometimes be possible to achieve very high transfection efficiencies using electroporation, the large number of critical parameters shows that this particular method has to be adjusted very accurately to the experimental conditions. The preliminary experiments which are required for optimizing the transfection by electroporation are made yet more difficult by the fact that a very wide variety of electroporation appliances, which markedly differ from each other with regard to the parameters which can be set, are available on the market.

An alternative method for introducing external nucleic acid into cells is based on directly microinjecting DNA into the cell nucleus (Capecchi (1980) Cell 22:479). This method has the advantage that the external DNA does not come into contact with cellular compartments, such as endosomes, which have a low pH which can chemically modify the DNA which has been introduced. However, since, in microinjection, each cell has to be transfected individually with DNA, this method is in no way suitable for any large-scale application, which is in fact what is required for biochemical analyses. The use of microinjection as a transfection method therefore remains restricted to the research laboratory and is only employed when specific problems require this.

In addition, artificial membrane vesicles (liposomes) have been investigated intensively for introducing external nucleic acid into cells, and it has been established that these transport vesicles are suitable for introducing DNA of interest into cells both in vitro and in vivo. In this method, the external DNA or RNA is enclosed in liposomes as a result of its negative overall charge. Subsequently, the liposomes are mixed with the cells to be transfected, resulting in the outside of the liposomes fusing with the cell membranes, due to their similarity with these membranes, and the nucleic acid being conveyed into the interior of the target cell (review article: Mannino and Gould-Fogerite (1988) BioTechniques 6:682). Although this method is both simple to carry out and leads to good results with regard to transfection efficiency, it cannot be used on an industrial scale due to the high cost of the liposomes which are required.

However, the method which is by far the most frequently used for introducing external nucleic acid into cells is that of calcium phosphate (CaPi)-mediated transfection. As early as 1973, Graham and van der Eb observed that the uptake of DNA in cultured cells can be increased markedly when this DNA is present as a calcium phosphate/DNA coprecipitate (Graham and van der Eb (1973) Virology 52:456). In these initial experiments, adenovirus and SV40 DNA were introduced into cells which were growing in firm contact with the bottom of the culture vessel (adherent cells). For this, calcium was used at a concentration of 125 mM and DNA was used at a concentration of from 5 to 30 μg/ml, with the optimum formation of a CaPi/DNA coprecipitate being observed at a neutral pH of 7.05. While the precise mechanism of CaPi-mediated DNA transfection is unknown, as in the case of DEAE dextran, it is assumed that the transfected DNA is taken up into the cytoplasm of the host cell by means of endocytosis and is then conveyed from the cytoplasm into the cell nucleus. In addition to determining the critical concentration ranges of calcium and DNA, Graham and van der Eb had determined the optimum reaction time for forming the CaPi/DNA coprecipitate to be 20-30 min and the subsequent time for incubating the cells with the coprecipitate to be 5-24 h. These initial studies laid the foundation for developing an efficient transfection method, which has been modified in very many different ways by a very great variety of laboratories over a period of what is now more than 30 years. For example, an additional increase in transfection efficiency was achieved by introducing an additional glycerol shock (Parker and Stark (1979) J. Virol. 31:360) and/or a treatment with chloroquine (Luthman and Magnusson (1983) Nucleic Acids Res. 11:1295). The use of sodium butyrate as being advantageous for the expression of proteins by plasmid-encoded genes which possess an SV40 enhancer has also been described (Gormann et al. (1983a) Nucleic Acids Res. 11:7631; Gormann et al. (1983b) Science 221:551).

Within the context of the many and diverse modifications which have been made to the CaPi-mediated transfection of cells, the treatment with chloroquine, which prevents the degradation of the external DNA by lysosome hydrolases, has been combined with the use of a glycerol shock, which increases the uptake of the external DNA into the cells. Depending on the type of starting cells which are used, it is possible to routinely transfect 90% of the cultured cells simultaneously when CaPi is employed. For this reason, CaPi-mediated transfection is frequently the method of choice both for transiently expressing external DNA in a large number of cells and for establishing stable cell lines which, after the transfection, contain integrated copies of the external DNA.

CaPi-mediated transfection is particularly suitable for efficiently transfecting mammalian cells, which, after the transfection, express the external DNA stably over a large number of cell culture passages (Chen and Okayama (1987) Mol. Cell. Biol. 7:2745). In this protocol, the CaPi/DNA coprecipitate is formed slowly and stepwise on the cells during the incubation with the cells to be transfected in the cell culture medium. It was found to be critical, for efficient transfection, that the pH of the buffer employed be 6.95 and that the concentration of circular DNA be 20-30 μg per approx. 106 cells. However, it was only possible to achieve these results using circularly closed DNA (plasmid DNA) whereas it was not possible to use this method to transfect linear DNA into cells. For the CaPi-mediated transfection, the authors used exponentially growing cells which were trypsinized prior to the transfection and subsequently sown in cell culture dishes at a cell density of approx. 5×10⁵ cells per 75 cm². The cells were incubated overnight with 10 ml of a standard nutrient culture medium. On the following day, 10-30 μg of the plasmid DNA of interest were incubated, at room temperature for 10-20 min, with 0.5 ml of 0.25 M CaCl₂ and 0.5 ml of 2×BES-buffered salt solution (2×BBS) which contain 50 mM BES (pH 6.95), 280 mM NaCl and 1.5 mM Na₂HPO₄. The CaPi/DNA coprecipitate which formed over this period of time was added dropwise, in a quantity of 1 ml, to the cells in the cell culture medium. The cell culture dishes were then tilted gently in order to distribute the CaPi/DNA mixture uniformly in the medium. There then followed an incubation step of 15-24 h at 35° C. in a 2-4% CO₂ atmosphere. After that, the medium was removed and the cells were washed twice in nutrient culture medium, then treated with fresh nutrient culture medium and incubated once again for a further 24 h at 35-37° C. and under a 5% CO₂ atmosphere. For selecting stable transformants, the cells were distributed, in a ratio of 1:10, on new cell culture receptacles.

Although the authors described for the first time, by means of this method, both a decrease in the temperature of the first incubation phase from the conventional 37° C. down to 35° C. and a decrease in the CO₂ atmosphere from the conventional 5% down to 2-4%, preferably 3%, CO₂ (temperature shift; CO₂ shift), the use of their method remains restricted to the laboratory scale, and the method is not suitable for large-scale use.

In the methods described in the literature, it is generally assumed that cells which are present in culture should be kept in a nutrient culture medium and under an atmosphere which is greatly enriched in C0₂ (Taylor et al. (1973) In Vitro 7(5):295; Minshull and Strong (1985) Int. J. Biochem. 17(4):529). Air, which, as a gas mixture, surrounds the Earth as a mantle, is composed, aside from approx. 77% nitrogen and 21% oxygen, of approx. 1.2% water vapor, 0.9% argon, 0.01% hydrogen and 0.03% carbon dioxide (CO₂). (Der kleine Brockhaus Enzyklopa die [The small Brockhaus Encyclopaedia], in two volumes (1950) Wiesbaden, Germany).

A CO₂ concentration (3-10% CO₂) which is increased approx. 100-300 times as compared with normal air is regarded as being essential for an efficient growth of cells under cell culture conditions, which are provided, in a cell culture incubator, by supplying external CO₂ (Chen and Okayama, see above; Chen and Okayama (1988) BioTechniques 6:632; Wilson (1995) Analytical Biochemistry 226;212).

WO 96/07750 also describes a method for efficiently transfecting eukaryotic host cells with DNA by means of CaPi-mediated transfection. Although this international patent application describes a method by which CaPi/DNA coprecipitate particles which are as large as possible are obtained, standard culture conditions with regard to the CO₂ atmosphere employed (5% CO₂) are taken as the basis in this case as well. The concentration of Ca²⁺ and PO₄³⁻, the pH and the temperature (35° C.) are regarded as being particularly critical for the CaPi-mediated transfection. On the other hand, the size of the CaPi/DNA coprecipitate particles can be reduced by the pH of the nutrient culture medium being slowly lowered due to the CO₂ which is produced by the cells (formation of HCO₃⁻ by the CO₂ and H₂O which are present in the nutrient culture medium) and the endogenous lactic acid production by the cells. However, the small particle size of the CaPi/DNA coprecipitate which is reached in this way is only suitable for transfecting particular cell types, which means that this method cannot be used in a general manner for standard cell lines. Although this international patent application describes the large-scale use of CaPi-mediated DNA transfection into cells in a bioreactor having a volume of up to 40 1, external CO₂ has to be introduced into this reactor as well in order to permit efficient cell growth.

Consequently, all the large number of CaPi/DNA transfection methods which have been described in the prior art are based on the cultured cells being gassed with external CO₂, something which requires the use of special incubation chambers (incubators, bioreactors) which possess a controllable CO₂ inlet. However, it is precisely for large-scale use that it is necessary to create reaction conditions which, especially from the point of view of costs, are simple and not particularly elaborate.

The present invention is therefore based on the object of creating a method for efficiently introducing nucleic acid into a cell by means of CaPi-mediated transfection, which method can be carried out inexpensively on the industrial scale.

It has now been found, surprisingly, that nucleic acid can be efficiently introduced into cells using CaPi-mediated transfection, with the cells being cultured in a closed culture system without any gassing with external CO₂.

The present invention therefore relates to a method for introducing at least one nucleic acid into at least one cell using calcium phosphate (CaPi), which comprises the steps of:

(a) mixing said nucleic acid or nucleic acids with at least one reaction solution which contains Ca²⁺ and PO₄³⁻ ions;

(b) incubating the reaction solution(s), resulting in the formation of a precipitate which contains at least nucleic acid, calcium and phosphate;

(c) adding the precipitate to said cell;

(d) incubating the cell from step (c) under air in a closed system without additional carbon dioxide (CO₂), resulting in the nucleic acid or nucleic acids being taken up into the cell.

In another preferred embodiment, two or more different nucleic acids, in particular two or three different nucleic acids, are transfected one after the other. In this connection, particular preference is given to using fresh medium for a fresh transfection.

The fact that the incubation in step (d) takes place under air in a closed system without additional carbon dioxide (CO₂) ensures, in particular, that the CaPi-mediated transfection method can be carried out on the industrial scale under simple and inexpensive conditions.

Within the meaning of the present invention, the designations “calcium phosphate (CaPi)-mediated transfection” and “CaPi/DNA coprecipitate” are understood as meaning the introduction of external nucleic acid into a host cell using a coprecipitate which is composed of Ca²⁺ and PO₄³⁻ ions and the nucleic acid to be introduced. In this connection, the precipitate contains hydroxyapatite (having the approximate chemical formula of (Ca₅OH(PO₄)₃)₂).

Any type of RNA (mRNA, rRNA, etc.) and any type of DNA, preferably, however, genomic DNA, cDNA, linear DNA or plasmid DNA, can be used as the external nucleic acid. Particular preference is given to the use of circularly closed plasmid DNA which is isolated from transformed bacteria using standard methods.

Within the scope of the present invention, the nucleic acid, which is preferably plasmid DNA, is used in a quantity of approx. 0.2 μg-20 μg, preferably of approx. 1 μg-20 μg, in particular of approx. 2 μg-10 μg, especially, however, of approx. 6 μg, per approx. 106 cells. High transfection efficiencies are achieved within these specified quantities.

In a particularly preferred embodiment, the method according to the invention can be used to transfect two or more different external nucleic acids, in particular two or three different external nucleic acids, simultaneously or one after the other. To achieve this, the nucleic acids of interest are mixed and used for forming the CaPi/DNA coprecipitate. In this connection, particular preference is given to using two or three different plasmid DNAs (cotransfection).

It is furthermore particularly advantageous to adjust the pH of the reaction solution in step (b) at room temperature to approx. 6.8-7.1, preferably to approx. 6.86-7.05, in particular to approx. 7.05, thereby achieving the greatest transfection efficiency.

Preference is also given to carrying out the incubation of the reaction solution in step (b) at room temperature (approx. 18-24° C.) for approx. 5-30 min, preferably for approx. 10-20 min, in particular for approx. 15 min. This results in the formation of particles of the CaPi/DNA coprecipitate which are of sizes which have proven to be particularly advantageous for efficient transfection.

In another particularly preferred embodiment, the incubation of the cells in step (d) is carried out at approx. 37° C. in an incubator for approx. 20-72 h, preferably approx. 30-72 h, in particular approx. 24-48 h, especially approx. 40-48 h. This period of time has proven to be optimal for ensuring that the cells are able to “recover” from the treatment with CaPi/DNA coprecipitate, and the uptake of the external nucleic acid which has taken place, before they are used for further investigations of the external nucleic acid or of the expression of genes which are encoded on the nucleic acid.

In another embodiment, the external nucleic acid which has been taken up remains permanently in the transfected cell (stable transfection). In this connection, it is replicated, for example as an episomal, extrachromosomal DNA particle, in the cell using a suitable origin of replication and passed on to the daughter cell in a controlled manner during cell division.

Alternatively, the external nucleic acid which has been introduced can also, if it does not possess any origin of replication, integrate, in a directed or undirected manner, into the genome of the cell, as a result of which it is also stably retained, replicated together with the genome of the host cell, prior to cell division, and subsequently passed on to the daughter cell during cell division.

In an alternative embodiment, the external nucleic acid which has been taken up is only retained in the transfected cell. Since it does not possess any suitable origins of replication, it is not replicated and is not passed on selectively to the daughter cell during cell division (transient transfection). This alternative method is especially advantageous if genes which are encoded on the external nucleic acid are expressed in the host cell in order subsequently to be analyzed in further experiments. This alternative method is furthermore suitable for investigating genes whose gene products (proteins) are toxic for the cell such that the cell would not survive a further cell culture passage.

In a preferred embodiment, a eukaryotic cell, preferably a mammalian cell and in particular a human cell, such as a HeLa cell, is, in particular, used as the starting cell for the transfection.

Adherently growing cell culture cells, which grow in constant contact with the culture vessel, in particular in a “lawn” on the bottom of the culture vessel, can, in particular, be used as starting cells for the method according to the invention. Furthermore, the cells can grow adherently on microcarriers. Examples of preferred cell lines are epidermal cell lines such as HeLa cells or 293 cells.

However, in an alternative embodiment, it is also possible to use cells which grow, and reproduced, in the cell culture medium without having any constant contact with the culture vessel, such as blood cells or cells of the lymphatic system which do not require a cell-cell contact for growth, even in the natural organism, and whose growth is direction-independent. Cell lines which have been adapted to growth in suspension culture are also suitable.

The present invention also relates to a method for producing a transfected cell, with the at least one nucleic acid being introduced into at least one cell using CaPi, in accordance with the method according to the invention, and to the transfected cell which is obtained using the method according to the invention.

The transfected cell can be used, in particular, for preparing recombinant adeno-associated viruses (rAAVs). A detailed description of the preparation of the rAAVs using the method according to the invention is given in the examples, with the examples only being intended to further clarify the invention without restricting the latter to these examples.

### EXAMPLES

1. Materials employed

| | |
|---|---|
| HeLa-t cells | MediGene Master Cell Bank |
| 6-well plates | NUNC; Cat. No. 152795 |
| T25 cell culture flasks | NUNC; Cat. No. 136196 |
| T185 cell culture flasks | NUNC; Cat. No. 144903 |
| Cell factories | NUNC; Cat. No. 164327 |
| DMEM cell culture medium | Life Technologies; Cat. |
| | No. 41965-039 |
| Fetal calf serum (FCS) | PAA; Cat. No. A15-653 |
| FCS North America | Gibco; Cat. No. 10084-169 |
| L-glutamine | LifeTechnologies; Cat. |
| | No. 25030-024 |
| Antibiotic/antimycotic | PAA; Cat. No. P11-002 |
| (AB/AM) | |
| Gentamycin | Gibco; Cat. No. 15750-037 |
| CaCl₂ | Sigma; C-7902 |
| H₂O | Sigma; W-3500 |
| 0.2 μm air filter | NUNC; Gelman 4210 |
| 2 × BBS buffer | 50 mM BES, 280 mM NaCl, |
| | 0.75 mM Na₂HPO₄, 0.75 mM |
| | NaH₂PO₄, pH 6.8-7.1 |
| | optimized) |
| Luciferase reagent | Promega E1501 |
| Triton lysis buffer | Promega E1501 |
| Luciferase-measuring | Berthold Lumat LB 9501 |
| instrument | |

2. Plasmids Employed

| | |
|---|---|
| Helper plasmid pSVori | Chiorini et al. (1995) |
| | Hum Gene Ther 6(12): 1531 |
| Helper plasmid | DE19905501 |
| pUC″ rep/fs/cap″ (RBS) Δ37 | |
| Helper plasmid | DE19905501 |
| pUC″ rep/cap″ Δ37 | |
| Helper plasmid | DE19905501 |
| pUC″ rep/cap″ (RBS) Δ37 | |
| Transgene plasmid AAV- | DE19905501 |
| (B7.2free/GMCSF) | |
| pCI-luc plasmid | Example 5 |
| Transgene plasmid pAAV-GFP | Example 5 |
| Transgene plasmid pAAV-(B7.2) | Example 5 |
| Adenovirus 5 | ATCC |

3. Transfecting while Gassing with CO₂, with and without Temperature and CO₂ Shift

3×10⁵ HeLa-t cells were in each case sown, in 3 ml of DMEM complete medium (500 ml of DMEM+50 ml of FCS+5 ml of L-glutamine+5 ml of AB/AM), in a well of a 6-well plate and incubated for 16-24 h at 37° C./5% CO₂. All the transfections were carried out in triplicate, with a total of four different transfections being performed: with and without a shift in the temperature and the CO₂ atmosphere in combination with no change in the medium or a change in the medium. The medium was changed directly before adding the DNA/CaPi precipitates. When this was done, the old medium was replaced with 3 ml of fresh, ungassed, prewarmed complete medium.

Forming the DNA/CaPi Precipitate:

Per well, 6 μg of pCI-luc DNA were thoroughly mixed with 150 μl of 260 mM CaCl₂ and this mixture was then carefully mixed with 150 μl of 2×BBS. The solution was left to stand for 15 min at room temperature. After that, in each case 300 μl of DNA/CaPi mixture per well were rapidly pipetted into the 3 ml of medium in a well.

The samples without any temperature/CO₂ shift were incubated directly at 37° C./5% CO₂. By contrast, the samples in which there was a shift were incubated for 16-20 h at 35° C./3% CO₂ and only after that at 37° C./5% CO₂.

After a total of approx. 40-48 h of incubation, the cells were harvested and expression of luciferase was measured. For this, the cells were washed with 500 μl of PBS, lyzed at room temperature in 500 μl of Triton lysis buffer and released from the surface using a cell scraper. The lysate was transferred to a 1.5 ml centrifuge tube and centrifuged at maximum rotational speed for 2 min. 2 μl of the supernatant were added to 50 μl of luciferase reagent and measured immediately.

The value which was measured was divided by the number of transfected cells (=sown-out cells×2) and multiplied by 1×10⁶ such that the value relates to [1×10⁶ cells.

TABLE 1
Comparison of a transfection with (+) and without (−) temperature/
CO₂ shift, without (−) and after (+) a change of medium. The luciferase
gene (luc) was used as the reporter gene. The measured values are
expressed in relative luciferase units (RLUs) per [1 × 10⁶ cells. The
percentage values indicate the transfection efficiency as compared with
the standard method (left-hand column = + shift − medium change =
100%). The blank values have already been subtracted. 3 experimental
assays were averaged in each case.

| | |
|---|---|
| +Temperature/CO₂ shift | −Temperature/CO₂ shift |

| −Medium | +Medium | −Medium | +Medium |
|---|---|---|---|
| change | change | change | change |
| 9.12 × 10⁸ | 7.15 × 10⁸ | 6.93 × 10⁸ | 7.49 × 10⁸ |
| 100% | 78% | 76% | 82% |

This experiment makes it clear that the standard method, with a temperature/CO₂ shift and no change in medium, gives a transfection efficiency which is similar to that of the other experimental mixtures (see Table 1).

4. Transfecting with and without CO₂ Gassing and with and without a Temperature Shift

HeLa-t cells were cultured, at 37° C./5% CO₂, in DMEM complete medium (500 ml of DMEM+50 ml of FCS+5 ml of L-glutamine+5 ml of AB/AM). 5×10⁵ cells were sown, per T25 flask, in 5 ml of complete medium. The cells were incubated at 37° C./5% CO₂ for 16-24 h. After that, the old medium was replaced with 3 ml of fresh, ungassed, warmed medium.

Preparing the DNA/CaPi Mixture:

Per T25, 6 μg of DNA were mixed with 150 μl of 260 mM CaCl₂ and this mixture was subsequently mixed carefully with 150 μl of 2×BBS. The solution was left to stand for 15 min at room temperature. After that, in each case 300 μl of the DNA/CaPi mixture were rapidly pipetted, per T25 flask, into the 3 ml of medium in the flask.

The samples in which there was no temperature shift and no gassing with CO₂ were incubated in an incubator at 37° C. and without any gassing with CO₂. For this, the caps were additionally sealed with three layers of parafilm. The samples in which there was a temperature shift were incubated for 16-20 h at 35° C./3% CO₂ and only after that at 37° C./5% CO₂.

After a total of approx. 40-48 h of incubation, the cells were harvested and luc expression was measured. For this, the cells were washed with 2000 μl of PBS, lyzed at room temperature in 1000 μl of triton lysis buffer and released from the surface using a cell scraper. The lysate was transferred to a 1.5 ml centrifuge tube and centrifuged at maximum rotational speed for 2 min. 2 μl of supernatant were added to 50 μl of luciferase reagent and measured immediately.

The measured value was divided by the number of the transfected cells (=sown-out cells×2) and multiplied by 1×10⁶, such that a value which is related to [1×10⁶ cells is obtained.

TABLE 2
Comparison of transfections with (+) and without (−) temperature
shift and with (+) and without (−) gassing with CO₂. 2 independent
experiments were carried out using in each case 3 averaged experimental
assays. Cells without CO₂ gassing were kept CO₂-free during the
transfection. The medium was changed before each of the transfections.
The measured values are in each case expressed in RLUs per 1 × 10⁶
cells. The values obtained with CO₂ and a shift were set to be equal
to 100% (= standard)

| | |
|---|---|
| Experiment 1 | Experiment 2 |

| +CO₂ | −CO₂ | +CO₂ | −CO₂ |
|---|---|---|---|
| +Temp. shift | −Temp. shift | +Temp. shift | −Temp. shift |
| 5.64 × 10⁸ | 5.06 × 10⁸ | 2.54 × 10⁸ | 2.76 × 10⁸ |
| 100% | 90% | 100% | 109% |

These results show that transfection without CO₂ gassing and without a temperature shift is just as efficient as the conventional method with CO₂ gassing and with a temperature shift (see Table 2).

5. Cloning the Plasmids:

1) pCI-luc was cloned as follows:

The vector pBL (Hoppe-Seyler et al. (1991) J. Virol. 65:5613-; Butz and Hoppe-Seyler (1993) J. Virol. 67:6476) was cut with the restriction enzymes SmaI and Pvu II in order to isolate the luciferase gene (insert). The vector pCI (Promega GmbH, Mannheim, Germany) was linearized with SmaI and dephosphorylated (vector). The two fragments, i.e. insert and vector, were ligated to give pCI-luc.

2) PAAV-GFP was prepared as follows:

The starting plasmid for subcloning the green fluorescence protein (GFP) was the vector pEGFP-N3 (Clontech, Heidelberg, Germany). A fragment of about 3000 bp in size was amplified by the polymerase chain reaction (PCR) using the oligonucleotides (primers) GFP/SpeI and GFP/MluI and subsequently cut with SpeI and MluI. This fragment was cloned into the basic AAV vector, resulting in pAAV-GFP being generated. The preparation of the basic vector is described in DE19905501. (Briefly, pAV2 (Laughlin et al. (1983) Gene 23:65) was cut with Bgl II and the AAV fragment in pUC19 was inserted by way of the BamHI cleavage site. In the resulting pUCAV2 plasmid, the AAV nucleotides 192-4497 were deleted and were replaced with the pCI nucleotides 4002-4008 and 1-1351, which contain the CMV-polyA expression cassette. This construct is termed the basic AAV vector).

Primer sequences:

| | |
|---|---|
| GFP/SpeI: | 5′-GGG ATC CAT CAC TAG TAT GGT GAG CAA |
| | GG-3′ |
| GFP/MluI: | 5′-CAA ACG ACC CAA CAC CAC GCG TTT TAT |
| | TCT GTC-3′ |

3) pAAV-(B7.2) was prepared as follows by way of two cloning steps:

The vectors pCI and pLL279 (Lanier et al. (1995) J. Immunol 154(1):97) were first of all cut with the restriction enzymes Xho I and Not I and the B7.2 insert from pLL279 was ligated to the pCI vector to give pC1-B7.2. A fragment of about 3000 bp in size was amplified from pC1-B7.2 by PCR using the primers B7.2/Nhe I and B7.2/Mlu I and subsequently cut with NheI and Mlu I. This fragment was cloned into the basic AAV vector (see Example 5.2), resulting in the generation of pAAV-(B7.2).

Primer sequences:

| | |
|---|---|
| B7.2/NheI: | 5′-GCA TTT GTG CTA GCA CTA TGG GAC TGA |
| | G-3′ |
| B7.2/MluI: | 5′-CGG TTC ACG CGT ATC AAG GCG ACT TAC |
| | ATC-3′ |

6. Packaging rAAV-GFP (Cell Culture in T25 cell Culture Flasks without any CO₂ Gassing)

HeLa-t cells were cultured in DMEM complete medium (500 ml of DMEM+50 ml of FCS+5 ml of L-glutamine+5 ml of AB/AM) at 37° C./5% CO₂. 5×10⁵ cells were seeded, per T25 cell culture flask (T25), in 5 ml of complete medium. The cells were incubated for 16-24 h at 37° C./5% CO₂. After that, the old medium was replaced with 3 ml of fresh, ungassed, warmed medium.

Preparing the DNA/CaPi Mixture (for Cotransfection):

Per T25, 2 μg of PAAV-GFP DNA were mixed with 4 μg of pSVori DNA and 150 μl of 260 mM CaCl₂ and this mixture was subsequently carefully mixed with 150 μl of 2×BBS. The solution was left to stand for 15 min at room temperature. After that, 300 μl of the DNA/CaPi mixture were pipetted rapidly, per T25, into the 3 ml of medium in the flask.

The samples without temperature shift and without CO₂ gassing were incubated in an incubator at 37° C. and without any gassing with CO₂. For this, the caps were additionally sealed with two layers of parafilm. The other mixtures were incubated for 16-20 h at 35° C./3% CO₂ and only after that at 37° C./5% CO₂.

After a total of approx. 40-48 h of incubation, the cells were infected with adenovirus. For this, the medium was removed from the flasks and replaced with 1.5 ml of fresh complete medium. The adenovirus solution was diluted down to 2×10⁹ particles/ml. 50 μl of the diluted virus were added to the 1.5 ml of medium in each flask. The flasks were sealed and appropriately incubated for 2 h. After that, 3 ml of fresh complete medium were added and the flasks were incubated for a further 20 h. The old medium was once again replaced with 5 ml of new medium.

Subsequently, the flasks were incubated for a further 60-70 h (appropriately depending on whether with or without CO₂, that is without and with an additional parafilm seal). After that, the cells and the supernatant were harvested. For this, the last, still adhering cells were rinsed off the bottom of the flask with supernatant and transferred into a 15 ml pointed-bottom centrifuge tube. Finally, the cells were lyzed by being frozen and thawed (in liquid N₂ and at 37° C. in a waterbath, respectively) three times.

Determining the Titer:

In order to determine the titer, 1.5×10⁵ HeLa-t cells were in each case sown in a well of a 12-well plate and incubated for 16-20 h (37° C./5% CO₂) in 1 ml of complete medium. After that, the cells were inactivated with y-radiation or UV radiation. The virus lysate was heat inactivated at 60° C. for 10 min, then centrifuged, and the supernatant was used for the virus titration. For this, the cells were infected with different quantities of the lysate and incubated at 37° C./5% CO₂ for 40-48 h. After that, the cells were harvested, with the supernatant being removed and the cells being washed with PBS and released from the cell culture vessel by being treated with EDTA/trypsin. Finally, the percentage of GFP-positive cells, and thus the virus titer, was determined by means of fluorescence-activated cell sorting (FACS).

TABLE 3
The cells were cotransfected with the transgene plasmid pAAV-GFP
and the helper plasmid pSVori as described under 4 (n = 39). The
further packaging took place in a standrad manner, but at −CO₂
−temp. shift and, furthermore, without CO₂ gassing. The values for
the standard were set at 100%. The measurement which was determined
was the quantity of virus produced per cell.

| | +CO₂ | −CO₂ |
|---|---|---|
| | +Temp. Shift | −Temp. Shift |
| Transducing particles | 12 | 14 |
| per cell | | |
| Performance criterion | 100% | 117% |

Accordingly, the efficiency of the packaging of rAAV-GFP without CO₂ gassing and without a temperature shift is comparable to that with CO₂ gassing and with a temperature shift (see Table 3).

7. Comparing Packaging in Cell Factories (CFs) with and without CO₂

HeLa-t cells were cultured in T185 cell culture flasks at 37° C./5% CO₂. The cells were harvested with EDTA/trypsin and resuspended in DMEM complete medium (500 ml of DMEM+50 ml of FCS+5 ml of 200 mM L-glutamine) (it is possible to add 50 μg of gentamycin/ml). From 0.9×10⁸ to 1.4×10⁸ HeLa-t cells were taken up in 900 ml of complete medium. A cell factory was filled with this cell suspension. For transfecting without CO₂ gassing and without a temperature shift, the two cell factory connections were sealed with parafilm or an air filter attachment which could be sealed in an airtight manner. The incubation took place at 37° C. for 22-30 h without CO₂. The assays with CO₂ gassing and with a temperature shift were incubated at 37° C./5% CO₂ for the same period of time.

After the incubation, the supernatant was removed. The first transfection with the relevant helper plasmid then took place (see Table 4). For this, 1800 μg of DNA were dissolved in 45 ml of 270 mM CaCl₂ and thoroughly mixed. After that, 45 ml of 2×BBS were added and mixed carefully. The DNA/CaPi mixture was left to stand for 20±5 min at room temperature. The 90 ml of DNA/CaPi solution were then rapidly added to 900 ml of prewarmed DMEM complete medium (with or without gentamycin). The medium, together with the precipitates, was transferred to the cell factory and the cells were incubated once again at 37° C. for 20-30 h without gassing.

After that, the relevant transgene plasmid (see Table 4) was transfected. This took place precisely as described for the helper plasmid.

TABLE 4
Combinations of helper plasmids and transgene plasmids
used in the experiments shown in Table 5 (see below).

| | | Number of |
|---|---|---|
| Helper plasmid | Transgene plasmid | experiments |
| PUC″ rep/fs/cap″ | pAAV-GFP | 1 |
| (RBS) Δ37 | | |
| PUC″ rep/fs/cap″ | pAAV-(B7.2) | 1 |
| (RBS) Δ37 | | |
| PUC″ rep/cap″ | pAAV- | 1 |
| (RBS) Δ37 | (B7.2free/GMCSF) | |
| PUC″ rep/cap″ Δ37 | PAAV- | 2 |
| | (B7.2free/GMCSF) | |

After 20-30 h of incubation, adenovirus was added to the cells at a multiplicity of infection (MOI) of 2-3. After a further 20-30 h of incubation at 37° C. and without gassing, the supernatant was replaced with 900 ml of fresh medium without FCS. After this last medium change, the cell factory was incubated at 37° C. for a further 60-68 h. The virus was then harvested by freezing/thawing lysis of the cells.

In comparison with this, no medium change took place, in the standard method, before transfecting with the helper plasmid. In addition, the cells were at the same time gassed through an 0.2 μm air filter. After the DNA/CaPi mixture had been added, the cells were first of all incubated overnight at 35° C./3% CO₂ before they were shifted to 37° C./5% CO₂. The two methods were identical apart from these differences.

TABLE 5
Comparison of cell factories (CFs) which were
used for packaging various vectors with (+) and without
(−) CO₂ gassing/temperature shift. CFs with and without
CO₂/temperature shift were compared directly with each
other in five experiments (= otherwise identical and
simultaneous preparation). The quantity measured was
the number of transducing particles per cell factory.

| | +CO₂ | −CO₂ |
|---|---|---|
| | +Temp. Shift | −Temp. Shift |
| Number of experiments | 5 | 5 |
| Average number of | 9.75 × 10⁹ | 10.01 × 10⁹ |
| transduced particles/CF | | |

The yields obtained when preparing recombinant AAVs without CO₂ gassing and without a temperature shift are also comparable with those obtained using the standard method, with CO₂ gassing and with a temperature shift, in cell factories, which are suitable for the industrial-scale culture of adherently growing cells (see Table 5).

1. A method for introducing at least one nucleic acid into at least one cell using calcium phosphate (CaPi), which comprises the steps of:
(a) mixing said nucleic acid or nucleic acids with at least one reaction solution which contains Ca²⁺ and PO₄³⁻ ions;
(b) incubating the reaction solution(s), resulting in the formation of a precipitate which at least contains nucleic acid, calcium and phosphate;
(c) adding the precipitate to said cell;
(d) incubating the cell from step (c) under air in a closed system without additional carbon dioxide (CO₂), resulting in the nucleic acid or nucleic acids being taken up into the cell.

2. The method according to claim 1, characterized in that the nucleic acid employed is RNA or DNA, preferably genomic DNA, cDNA, linear DNA or plasmid DNA, in particular circular plasmid DNA.

3. The method according to claim 1 or 2, characterized in that the nucleic acid is used in a quantity of 0.2 μg-20 μg, preferably of 1 μg-20 μg, in particular of 2 μg-10 μg, especially of 6 μg, per 10⁶ cells.

4. The method according to one of claims 1 to 3, characterized in that two or more different nucleic acids, preferably two or three different nucleic acids, in particular two or three different plasmid DNAs, are used simultaneously or one after the other.

5. The method according to one of claims 1 to 4, characterized in that the pH of the reaction solution in step (b) is, at room temperature, 6.8-7.1, preferably 6.86-7.05, in particular 7.05.

6. The method according to one of claims 1 to 5, characterized in that the incubation of the reaction solution in step (b) is carried out at room temperature for 5-30 min, preferably for 10-20 min, in particular for 15 min.

7. The method according to one of claims 1 to 6, characterized in that the incubation of the cells in step (d) is carried out at 37° C. for 20-72 h, preferably 30-72 h, in particular 24-48 h, especially 40-48 h.

8. The method according to one of claims 1 to 7, characterized in that the nucleic acid which has been taken up has an origin of replication, is retained episomally in the transfected cell and is passed on, in a controlled manner, to the daughter cell during cell division.

9. The method according to one of claims 1 to 7, characterized in that the nucleic acid which has been taken up does not have any origin of replication and integrates, in a directed or undirected manner, into the genome of the cell and is thereby passed on to the daughter cell during cell division.

10. The method according to one of claims 1 to 7, characterized in that the nucleic acid which has been taken up does not have any origin of replication and is consequently not selectively passed on to the daughter cell during cell division.

11. The method according to one of claims 1 to 10, characterized in that the cell employed is a eukaryotic cell, preferably a mammalian cell, in particular a human cell.

12. The method according to one of claims 1 to 11, characterized in that the cell grows, and reproduces, in the cell culture medium in constant contact with the culture vessel.

13. The method according to one of claims 1 to 11, characterized in that the cell grows and reproduces in the cell culture medium without any constant contact with the culture vessel.

14. A method for preparing a transfected cell, characterized in that at least one nucleic acid is introduced into at least one cell using CaPi in accordance with the method according to one of claims 1 to 13.

15. A transfected cell which can be obtained by the methods according to one of claims 1 to 14.


SEQUENCE LISTING (4 sequences)

SEQ ID NO: 1 - DNA, 29 bp, Artificial Sequence
  Primer
gggatccatcactagtatggtgagcaagg

SEQ ID NO: 2 - DNA, 33 bp, Artificial Sequence
  Primer
caaacgacccaacaccacgcgttttattctgtc

SEQ ID NO: 3 - DNA, 28 bp, Artificial Sequence
  Primer
gcatttgtgctagcactatgggactgag

SEQ ID NO: 4 - DNA, 30 bp, Artificial Sequence
  Primer
cggttcacgcgtatcaaggcgagttacatg